(19) United States
(12) Patent Application Publication
(10) Pub. No.: US 2009/0197293 A1
(43) Pub. Date: Aug. 6, 2009

(54) NOVEL USE OF AN INDICATOR CELL LINE FOR BIOASSAY

(75) Inventors: Nandan M. CHANDAVARKAR, Mumbai (IN); Amit C. TAWTE, Mumbai (IN); Anagha A. SHIRKE, Mumbai (IN); Mariamma A. PARACKAL, Thane (IN)

Correspondence Address: BLANK ROME LLP, WATERGATE, 600 NEW HAMPSHIRE AVENUE, N.W., WASHINGTON, DC, 20037, US

(73) Assignee: FDC Limited, Mumbai (IN)

(21) Appl. No.: 12/194,947

(22) Filed: Aug. 20, 2008

(30) Foreign Application Priority Data
Feb. 6, 2008 (IN) 267/MUM/2008

(51) Int. Cl. C12Q 1/02

(52) U.S. Cl. 435/29

(57) ABSTRACT

The present invention relates to methods of bioassay for detecting the effective concentration of Granulocyte Colony Stimulating Factor (G-CSF), and estimating ED₅₀ and specific activity of G-CSF using a human acute myeloid cell line. The invention also relates to use of this technique for estimating the potency of G-CSF samples during various stages of a purification process.

This application claims the priority of Indian Provisional Patent Application No. 267/MUM/2008, filed Feb. 6, 2008.

### FIELD OF THE INVENTION

The present invention relates to methods of bioassay for detecting the effective concentration of granulocyte colony stimulating factor, and estimating ED₅₀ and specific activity of G-CSF using a human acute myeloid cell line. The invention also relates to use of this technique for estimating the potency of G-CSF samples during various stages of a purification process.

### BACKGROUND OF THE INVENTION

Various methods available for determination of protein concentration, purity, and structural configuration can be applied to G-CSF preparations as well. General protein estimation methods, such as Lowry's, Biuret or absorbance assay in the UV range, estimate the total protein concentration of purified sample. Methods such as, SDS PAGE and RP-HPLC, are used to determine the purity and concentration of proteins in the sample. Specific methods, such as G-CSF ELISA, are available to estimate the concentration, as well as binding properties (and hence the potency) of a molecule (e.g. G-CSF) to its receptor. Methods, such as MALDI-TOF mass spectroscopy and CD-spectroscopy, can reveal, in detail, the structural configuration and integrity of the molecule required for its proper biological activity.

However, none of these methods can estimate the biological activity of G-CSF in a sample, as none of them takes into account the actual functional properties of the G-CSF molecule. The methods mentioned above are based on properties such as structure, molecular weight, ionic charge, and purity of the molecule. The biological activity of the sample can only be estimated by in vivo (animal based) or in vitro (cell based) bioassays.

In vivo, the G-CSF molecule stimulates the survival and proliferation of myeloid progenitor cells, as well as their differentiation and maturation towards neutrophilic granulocytes. In addition, G-CSF stimulates the release of mature neutrophils from bone marrow and brings about their activation. An in vivo bioassay of G-CSF makes use of this fact by measuring the increase in number of circulating neutrophils in the blood of injected neutropenic mouse. As for all animal based bioassays, this in vivo assay suffers limitations, such as use of animals, variations in the results due to variations in the animal models, etc.

In vitro bioassays are used for determining the biological activity of cytokines. These assays are not only more sensitive than immunoassays, but are the only techniques to determine biological activity without making use of any animal models. A bioassay is useful in detecting factors acting on early myeloid progenitors and, hence for factors potentially useful as haemopoietic cell regulators. The assay can be conducted using relatively crude or semi-purified samples such as condition medium or purified known or unknown factors (U.S. Pat. No. 5,322,787 which is incorporated herein by reference). For this reason, in vitro bioassays are indispensable for characterizing and studying any cytokine. Also in vitro bioassays are intended to be used for quality control and illustrate the batch to batch consistency of biological potency of a product (A. R Mire Sluis, et al. “Quality consideration for recombinant DNA-derived biological therapeutic products”, Bio drugs Vol. 11, pp. 367-376, 1999).

In case of G-CSF, many cell lines are available that respond to G-CSF. Examples of such cell lines are NFS-60 (Shawn DeFrees et al. “GlycoPEGylation of recombinant therapeutic proteins produced E. coli, Novel G-CSF Conjugates”, Oxford Journals Glycobiology, Vol. 16, No. 9, pp. 833-843, 2006.), M-NFS-60 (Shin-Young Honga et al. “Production of bioactive human granulocyte stimulating factor in transgenic rice cell suspension cultures”, Protein Expression and Purification, Vol. 47, pp 168-73, 2006), AML-193 (Hiroya Asou et al. “Establishment of Human Acute Myeloid Leukemia Cell Line Kasumi-1 with 8; 21 Chromosome Translocation”, Blood, Vol. 77, No. 9, 1991 pp 2031-2036), WEHI-3B cells (Nicosa. Nicola et al. “Binding of differentiation—inducer granulocyte colony stimulating factor to responsive but not so responsive leukemic cell lines”, Proc. NaH. Acad sci. USA Vol 81, 1984 pp 3765-3769), HL-60 (Yamaguchi T et al “Bioassay of human granulocyte colony stimulating factor using promyelocytic HL-60 cells”, Biological and pharmaceutical Bulletin 1997, vol. 20, No 9, pp. 943-947; Ilona Nakoinz et al., “Differentiation of IL-3 Dependent NFS-60 Cell Line and Adaption to growth in Macrophage Colony Stimulating Factor”, Journal of Immunology, Vol. 145, Issue 3, pp. 860-864, 1990), etc. Out of these cell lines, the murine myeloblastic cell line NFS-60 is a commonly used cell line for the bioassay and potency estimation of G-CSF in various samples. The cell line requires IL-3 for its growth and continual maintenance and also responds to G-CSF and GM-CSF (Ilona Nakoinz et al., “Differentiation of IL-3 Dependent NFS-60 Cell Line and Adaption to growth in Macrophage Colony Stimulating Factor”, Journal of Immunology, Vol. 145 Issue 3, pp. 860-864, 1990). However, this cell line is not available with the most important cell repository, the American Type Culture Collection (ATCC).

Another cell line is derived from IL-3 dependent murine cell line NFS-60 by growing the cell line in presence of 10% L cell conditioned medium as a source of M-CSF or pure M-CSF for a few continual passages. The subline derived, called M-NFS-60, is available with ATCC. This cell line requires M-CSF (62 ng/mL) for growth and maintenance but fails to grow in absence of M-CSF. It gives proper response to M-CSF and IL-3 but a very weak response to G-CSF (Ilona Nakoinz et al.). When this cell line was used for bioassay of G-CSF, the response of cell line for G-CSF was very weak. Also, the response is not consistent over the concentration range of G-CSF, but shows a variation after few passages. Because of this, the results of G-CSF bioassay were not consistent, and the method could not be used confidently for potency estimation of G-CSF samples. Also, the presence of M-CSF in regular maintenance and assay medium of M-NFS-60 cell line hinders the bioassay of G-CSF and complicates the result analysis. Therefore, there remains a need to develop a simple method for G-CSF bioassay.

### SUMMARY OF THE INVENTION

Accordingly, the present invention relates to the use of a human acute myeloid cell line, Kasumi-1 (ATCC No. CRL-2724), for G-CSF bioassay. Kasumi-1 cell line is derived from the peripheral blood of a 7-year-old Japanese boy with Acute Myeloid Lymphoma (AML) in relapse after bone marrow transplantation. The cell line was procured from ATCC. The particular cell line was selected for G-CSF assay development because, as per ATCC's product description, the cell line shows response to IL-3, IL-6 GM-CSF and G-CSF. The cell line shows neither granulocytic nor eosinophilic maturation in vitro liquid culture by addition of DMSO, G-CSF or IL-5 (Hiroya Asou et al. “Establishment of Human Acute Myeloid Leukemia Cell Line Kasumi-1 with 8:21 Chromosome Translocation”, Blood, Vol. 77, No. 9, 1991 pp 2031-2036). The most important properties considered for selection of this cell line were as follows 1) responsiveness to G-CSF; 2) no compulsory requirement of any cytokine for its growth and its maintenance in media routinely; 3) minimal medium requirement to maintain the cell line; and 4) its human origin. The cell line, after being obtained from ATCC, was expanded in the laboratory to get the sufficient number of token, master and working stocks. These stock cultures were then used for the various experimental procedures, which led to the standardisation of a bioassay procedure for G-CSF.

An object of the present invention relates to methods of bioassay of G-CSF by making use of a more stable, easy to maintain cell line Kasumi-1 as an indicator cell line for G-CSF bioassay. In the methods, Kasumi-1 cell line is used to establish the G-CSF bioassay in order to estimate the potency of various samples of G-CSF in terms of ED₅₀ and specific activity, which are obtained, e.g. during purification process of the protein. As used herein, “potency” refers to the measure of biological activity using a suitably quantitative biological assay based on the attribute of the product that is linked to the relevant biological properties. For example, in the case of G-CSF, the biological activity is the ability of G-CSF to stimulate the growth of Kasumi-1 cell line. To determine the specific activity of G-CSF in a sample, a non-linear graph is plotted using log concentration of G-CSF v/s Absorbance in terms of Optical density for Cell Growth at 570 nm. A dose response curve is obtained which is used to calculate the ED₅₀ values from the activity plot. An ED₅₀ value is the amount of rh-G-CSF that promotes the cell growth of 50%, i.e. 50% absorbance, against the maximum cell growth, i.e the maximum absorbance (U.S. Patent Application Publication No 2007/0166278 A1, which is incorporated herein by reference). ED₅₀ value and rhG-CSF activity are inversely proportional to each other therefore, lowest is the ED₅₀ value highest is the rhG-CSF activity.

In accomplishing the foregoing objectives, the bioassay comprises the steps of: (a) contacting a sample containing G-CSF with Kasumi-1 cells; (b) determining the growth of the Kasumi-1 cells in the presence of the sample; and (c) correlating the growth of the Kasumi-1 cells to an activity of G-CSF. Step (a) preferably involves adding the sample containing G-CSF to a cell culture containing Kasumi-1 cells. Step (b) preferably involves incubating the sample with the Kasumi-1 cell culture for a predetermined period of time to allow the cells to grow under the influence of the G-CSF. At the end of the predetermined period, the cell density of the culture is measured, by using, for example, an indicator dye such as MTT. Step (c) preferably involves comparing the cell density of the culture measured in step (b) with a standard dose-response curve to determine the activity of the G-CSF.

The Bioassay may conveniently be provided in a kit form. According to this aspect of the present invention there is provided a kit for carrying out the bioassay of G-CSF using Kasumi-1 cells. This kit includes, in packaged combination, (a) a first container containing a culture of Kasumi-1 cells; and (b) a separate container containing a known amount of G-CSF. The kit may also include a container(s) containing dilution solution(s), along with instructions for performing the bioassay, and multi-well plates

Yet another object of the present invention provides a method for determining the ED₅₀ of a sample containing G-CSF. To determine ED₅₀, A Non-linear logarithmic graph is plotted using concentration of rh-GCSF v. growth observed, e.g. as absorbance at about 570 nm. Non-linear regression analysis provides us with the ED₅₀ values.

Other objects, features and advantages of the present invention will become apparent to those skilled in the art from the following detailed description. It is to be understood, however, that the detailed description and specific examples, while indicating preferred embodiments of the present invention, are given by way of illustration and not limitation. Many changes and modifications within the scope of the present invention may be made without departing from the spirit thereof, and the invention includes all such modifications.

### BRIEF DESCRIPTION OF FIGURES

FIG. 1 is a graphical representation of growth of M-NFS-60 in presence and in absence of M-CSF and G-CSF. M-NFS-60 cells were washed three times in PBS and seeded to medium containing M-CSF, G-CSF, M-CSF+G-CSF and no cytokine at a density of 1×10⁵ cells/mL. Flasks incubated for 6 days and response of M-NFS-60 to these conditions was estimated by reduction of Alamar Blue incorporated in growth medium.

FIG. 2 is a graphical representation of growth of Kasumi-1 in presence and in absence of M-CSF and G-CSF. Kasumi-1 cells were washed three times in PBS and seeded to medium containing M-CSF, G-CSF, M-CSF+G-CSF and no cytokine at a density of 1×10⁵ cells/mL. Flasks were incubated for 6 days and response of Kasumi-1 to these conditions was estimated by reduction of Alamar Blue incorporated in growth medium.

FIG. 3 is a graphical representation of response of Kasumi-1 and M-NFS-60 to M-CSF and M-CSF+G-CSF. Kasumi-1 and M-NFS-60 cells were washed three times in PBS and seeded to medium containing M-CSF and M-CSF+G-CSF at a density of 1×10⁵ cells/mL. Flasks were incubated for 6 days and response of cells to these conditions was estimated by reduction of Alamar Blue incorporated in growth medium.

FIG. 4 is a graphical representation of response of Kasumi-1 and M-NFS-60 to G-CSF and M-CSF+G-CSF. Kasumi-1 and M-NFS-60 cells were washed three times in PBS and seeded to medium containing G-CSF and M-CSF+G-CSF at a density of 1×10⁵ cells/mL. Flasks were incubated for 6 days and response of cells to these conditions was estimated by reduction of Alamar Blue incorporated in growth medium.

FIG. 5 is a graphical representation of effect of seeding density of Kasumi-1 for G-CSF bioassay. Kasumi-1 cells were harvested and seeded at three different cell densities of 1×10⁵, 5×10⁵ and 1×10⁶ cells/mL.

FIG. 6 is a graphical representation of effect of diluent of Kasumi-1 for G-CSF bioassay. Kasumi-1 cells were harvested and seeded at a density of 5×10⁵ cells/mL. Protein sample diluted in complete medium and Elix water is added to the culture wells covering a concentration range of 10⁻² to 10⁶ pg/mL.

FIG. 7 is a graphical representation of effect of incubation time of Kasumi-1 for G-CSF bioassay. Kasumi-1 cells were harvested and seeded at a density of 5×10⁵ cells/mL in three different microtiter plates. Protein sample diluted in complete medium is added to the culture wells covering a concentration range of 10⁻² to 10⁶ pg/mL. Each of the three plates were harvested at respective time intervals of 24, 48 and 96 hrs.

FIG. 8 is a graphical representation of effect of volume of G-CSF dilution on Kasumi-1 for G-CSF bioassay. Kasumi-1 cells were harvested and seeded at a density of 5×10⁵ cells/mL. Protein sample prepared from three different stock solutions of 6 μg/mL, 3 μg/mL and 2.33 μg/mL and diluted in complete medium are added to the seeded plates in three volumes of 20 μL, 50 μL and 75 μL respectively covering a range of 10⁻² to 10⁶ pg/mL.

FIG. 9 is a graphical representation of average response of Kasumi-1 towards G-CSF. G-CSF bioassay is repeated multiple times under standardised conditions and the results obtained were converted to percentage of negative control. The averages of these negative controls were plotted to get the average response of Kasumi-1 cells to G-CSF in the concentration range of 10⁻² to 10⁶ pg/mL

FIG. 10A is a graphical representation of determination of specificity of Kasumi-1 cells towards rhG-CSF in presence of another cytokine M-CSF. Kasumi-1 cells were harvested and three different cell suspensions were prepared each at 5×10⁵ cells/ml. Suspension A contained no cytokine, suspension B contained G-CSF and suspension C contained M-CSF, both at a fixed concentration 10² pg/ml (Spiking). These suspensions are plated in two different microtiter plates and 50 μl of 10-fold serial dilution of G-CSF and M-CSF covering a range of 10⁻² to 10⁶ pg/ml were added to the plates in duplicates such that one plate contained only G-CSF (10⁻² to 10⁶ pg/ml) and M-CSF (10² pg/ml)+G-CSF (10⁻² to 10⁶ pg/ml) and another plate contained only M-CSF (10⁻² to 10⁶ pg/ml) and G-CSF (10² pg/ml)+M-CSF (10⁻² to 10⁶ pg/ml). Plates were harvested after 48 hrs. of incubation.

FIG. 10B is a graphical representation of determination of specificity of Kasumi-1 cells towards rhG-CSF in presence of yet another cytokine IL-4. Kasumi-1 cells were harvested and three different cell suspensions were prepared each at 5×10⁵ cells/ml. Suspension A contained no cytokine, suspension B contained G-CSF and suspension C contained IL-4, both at a fixed concentration 10² pg/ml (Spiking). These suspensions are plated in two different microtiter plates and 50 μl of 10-fold serial dilution of G-CSF and IL-4 covering a range of 10⁻² to 10⁶ pg/ml were added to the plates in duplicates such that one plate contained only G-CSF (10⁻² to 10⁶ pg/ml) and IL-4 (10² pg/ml)+G-CSF (10⁻² to 10⁶ pg/ml) and another plate contained only IL-4 (10⁻² to 10⁶ pg/ml) and G-CSF (10² pg/ml)+IL-4 (10⁻² to 10⁶ pg/ml). Plates were harvested after 48 hrs. of incubation.

FIG. 11a is a graphical representation of position effect on G-CSF bioassay using randomised plate design for rhG-CSF dilutions. Kasumi-1 cells are harvested and plated at 5×10⁵ cells/ml density in four different microtitre plates. Stock solution of rhG-CSF of 3 μg/ml is diluted covering a range of 10⁻² to 10⁶ pg/ml. 50 μl of the each G-CSF dilution prepared in media is then added to each plate in four different sets and duplicates of each. These dilutions are then plated in randomised plate design format as shown in FIG. 11b.

FIG. 11b shows the plate design formats used to determine the position effect.

FIG. 12 is a graphical representation of effect of different sera on the performance of Kasumi-1 cells for G-CSF bioassay, the cells are maintained and harvested from media containing two different sera and G-CSF bioassay is performed using these cells in complete media containing respective sera.

FIG. 13 is a graphical representation of effect of different methods of harvesting on rhG-CSF bioassay of Kasumi-1. Kasumi-1 cells were harvested and seeded at a density of 5×10⁵ cells/mL in three different microtiter plates. G-CSF is diluted in complete medium and is added to the culture wells covering a concentration range of 10⁻² to 10⁶ pg/mL. the assay is then harvested using three different growth indicators such MTT, resazurin based indicator Alamar Blue and LDH.

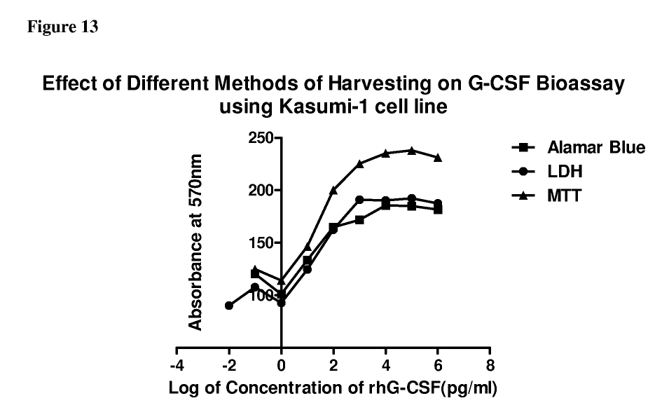

FIG. 14 is a graphical representation of comparison of efficiency of Kasumi-1 and M-NFS-60 cell line for bioassay of G-CSF. G-CSF bioassay is performed under standardised conditions for both the cell lines.

FIG. 15 is a graphical representation of efficiency of rhG-CSF sample purification; G-CSF samples are collected at various stages of purification and assayed using bioassay of Kasumi-1 cell line. Kasumi-1 cells were harvested and seeded at a density of 5×10⁵ cells/mL in microtitre plate. Stock solutions of various G-CSF samples obtained during various stages of purification, are prepared in preincubated sterile complete media each at the concentration of 3 μg/ml. Serial 10 fold dilutions are performed from these stock solutions in sterile complete media covering a range of 3×10⁻² to 3×10⁶ pg/ml for each sample. 50 μl of each dilution is then added in plate containing Kasumi-1 cells in duplicate. Plates were harvested after 48 hrs. of incubation.

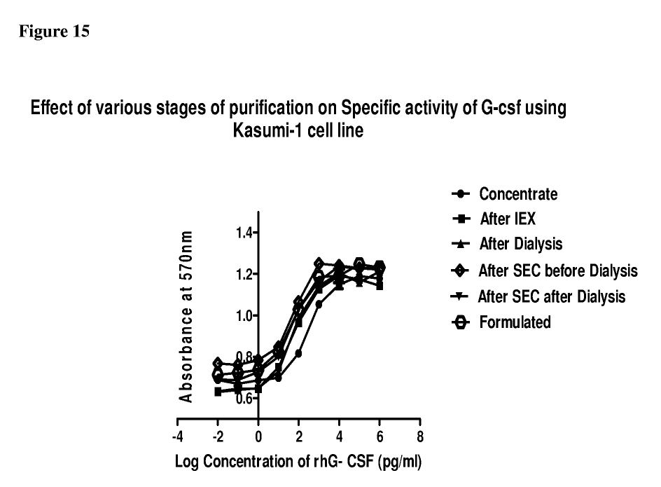

### DETAILED DESCRIPTION OF THE INVENTION

The following abbreviations are used throughout the specification:

M-CSF: Macrophage colony stimulating factor
G-CSF: Granulocyte colony stimulating factor
rhG-CSF: Recombinant Human Granulocyte colony stimulating factor
rhM-CSF: Recombinant Human Macrophage colony stimulating factor
GM-CSF: Granulocyte macrophage colony stimulating factor

### IL-3: Interleukin 3

### IL-5: Interleukin 5

### IL-6: Interleukin 6

ED₅₀: Effective dose 50
FBS: Fetal bovine serum
PBS: Phosphate buffered saline

### RPMI-1640: Rosewell Park Memorial Institute 1640

CO₂: Carbon dioxide
DMSO: Dimethyl sulphoxide

### ATCC: American Type Collection Centre

MTT: (3-(4,5-dimethylthiazolyl-2)-2,5-diphenyltetrazolium bromide
SDS PAGE: Sodium dodecyl sulphate polyacrylamide gel electrophoresis
RP-HPLC: Reverse phased high performance liquid chromatography

### UV: Ultraviolet

ELISA: Enzyme linked immunosorbant assay
MALDI-TOF: Matrix assisted laser desorption/ionisation time-of-flight
CD: Circular dichromism
RH: Relative humidity
RT: Room temperature
LDH: Lactate dehyrogenase

The invention will now be described in detail in connection with certain preferred and optional embodiments, so that various aspects there of may be more fully understood and appreciated.

The cell bioassay according to the present invention represents a significant improvement over the known assay methods. The inventive bioassay is simpler and faster than the standard mouse assay, and requires fewer samples than the mouse assay. Furthermore, the instant bioassay also can be carried out more easily than other previously known cell-based assays because it is simpler and more reliable.

According to an embodiment of the invention, there is provided a bioassay for G-CSF, which is useful for the estimation of potency of G-CSF in terms of ED₅₀ and specific activity. The assay preferably uses a human acute myeloid cell line, i.e. Kasumi-1. In a preferred embodiment, the G-CSF used is of human origin expressed as a recombinant protein in prokaryotic system. Preferably, a G-CSF of 95% or more purity is used for a bioassay and other experimental purposes in the present invention.

The cell cultures employed according to the inventive bioassay contains Kasumi-1 cells, which are suitable for use in the present assay. The cells also are responsive in a dose-dependent manner to G-CSF. The cells are also responsive to the selected indicator. The indicator employed in a bioassay within the instant invention preferably distinguishes active, living cells from dead cells, and is a substrate for mitochondrial dehydrogenase. Living cells act upon the indicator to produce a measurable product, while dead cells do not act upon the indicator to produce a measurable product. Preferred indicators include 3-(4,5-dimethylthiazol-2-yl)-2,5-diphenyl tetrazolium bromide (MTT), 2,3-bis(2-methoxy-4-nitro-5-sulfophenyl)-2H-tetrazolium-5-carboxanilide inner salt (XTT) (Sigma #X4251) (Parsons et al., J. Heterocyclic Chem. 25: 911 (1988); Scudievo et al., Cancer Res. 48: 4827 (1988)) and methylene blue. MTT is metabolized only in living cells, whose mitochondria cleave its tetrazolium ring to produce a blue-colored formazan product. XTT is metabolized only in living cells, whose mitochondria cleave its tetrazolium ring to produce an orange/red formazan product. Living cells decolorize methylene blue by cell dehydrogenase activity. The indicator is used to measure cell growth in response to exposure to G-CSF.

The bioassay comprises the steps of: (a) contacting a sample containing G-CSF with Kasumi-1 cells; (b) determining the growth of the Kasumi-1 cells in the presence of the sample; and (c) correlating the growth of the Kasumi-1 cells to an activity of G-CSF.

Step (a) preferably involves adding the sample containing G-CSF (preferably about 20-75 μL, most preferably 50 μL) to a cell culture containing Kasumi-1 cells. The sample and/or cell culture may be diluted prior to being mixed together. The sample should be diluted to be in the linear range. This normally requires serial dilution of the sample and adding the several dilutions to the cell culture. The cell culture should be diluted to contain the optimal cell concentration for the assay, which is preferably from about 1×10⁵ to about 5×10⁵ cells/mL, most preferably is 5×10⁵ cells/mL. The preferred dilution solution for the sample is pre-incubated sterile complete RPMI 1640 medium with 20% FBS (“preincubated” as used here in means that, prior to use, the medium has been stored at 5% v/v CO₂ in air at 37° C., 85% humidity overnight for obtaining the desired pH, and checked for sterility). The preferred dilution solution for the cells is pre-incubated sterile complete RPMI 1640 media with 20% FBS. The G-CSF sample/cell culture mixture is preferably incubated at about 37° C. with 85% relative humidity (RH), and 5% CO₂, for about 24-72 hours, most preferably for 48 hours.

Step (b) preferably involves incubating the sample with the Kasumi-1 cell culture for a predetermined period of time to allow the cells to grow under the influence of and in response to the G-CSF. At the end of the predetermined period, the cell growth of the culture is measured, by using, an indicator as mentioned above. Especially preferred indicator is MTT. The MTT assay is a standard colorimetric assay for measuring cellular growth well known in the art. Briefly, MTT is reduced to purple formazan in the mitochondria of living cells. Because this reaction only occurs when mitochondrial reductase enzymes are active, the color change can directly be correlated with the number of viable cells, and thus, cell growth. Because purple formazan is insoluble, a solubilization solution, preferably a surfactant (DMSO or SDS) or detergent available with the ATCC, MTT Cell Proliferation Kit (Cat. No. 30-1010K-A), is added to dissolve the insoluble purple formazan product into a colored solution whose absorbance can be measured in a spectrophotometer at a wavelength of between about 500 to about 600 nm, preferably about 570 nm. The absorbance is thus directly related to the number of viable cells, and thus, cell growth. The specific steps for using the MTT assay are as follows:
i. adding the MTT to the G-CSF with Kasumi-1 cells mixture of step (a);
ii. incubating the mixture of step i above, preferably for about 3-4 hours, and/or at about 37° C., about 5% CO₂, and about 85% relative humidity (RH);
iii. adding a detergent after the conclusion of step ii;
iv. incubating the mixture of step iii, preferably for about 16-18 hours, most preferably 18 hours; and
v. measuring the absorbance of the mixture of step iv at a wavelength of about 500-600 nm, preferably about 570 nm.

Steps (c) preferably involve comparing the cell growth of the culture measured in step (b) with a standard dose-response curve to determine the concentration or activity of the G-CSF. For example, if MTT is used as an indicator as discussed above, the measured absorbance can be correlated with the concentration or activity of the G-CSF in the sample by using a standard dose-response curve, which can be obtained by incubating known amounts of G-CSF with Kasumi-1 cells. Methods for obtaining standard (or calibration) curves are well-known in the art and is within the knowledge of a skilled artisan.

In a preferred embodiment of the invention, the effective amount of Kasumi-1 cells (subcultured 24-48 hrs. prior to assay) is added to RPMI 1640 medium with 20% v/v FBS and plated with 100 μL of cell suspension in each well of 96-well microtiter plate excluding the wells of all four edges (wells of the four edges are filled with 100 μl of complete media to maintain humidity and to avoid the edge effect). The effective amount of cells was standardised and kept close to 5×10⁵ cells/mL of complete medium. To these wells was then added, in duplicates, 50 μL of G-CSF dilution performed in complete growth medium covering a range of G-CSF concentration of 10⁻² to 10⁶ pg/mL of diluent (i.e. complete growth medium). The controls were set up in duplicates containing only cells and complete growth medium but no cytokine.

All the wells of edges were filled with 100 μL of medium only to protect from edge effect. The plate was then incubated in 5% v/v CO₂ in air at 37° C., 85% humidity for 48 hrs. The extent of Kasumi-1 cell proliferation due to amount of G-CSF in the given well was determined by using a redox indicator MTT. The graph of absorbance vs. G-CSF concentration was plotted and ED₅₀ and specific activity was determined using GraphPad Prism software (Version 5.00).

The methods of the present invention can be used alone or in conjunction with other assays for G-CSF, such as those previously mentioned in the Background of the Invention. These other assays may be, but are not limited to, immunoassay (e.g. ELISA), SDS-PAGE, HPLC, and spectroscopy (e.g. MALDI-TOF mass spectroscopy or CD-spectroscopy).

For convenience, kits can be provided for carrying out the inventive bioassay method. The kits may include the solutions utilized in the method of the invention, with all constituents in appropriate containers (vials, syringes, pipettes, etc.) in predetermined proportions and amounts. The kits also may include means for contacting the solutions with the fluid sample, such as multi-well plates or test tubes. Where applicable, the kits may include an appropriate solvent for formazan, such as SDS, and at least one calibration curve for G-CSF or sufficient amount of G-CSF to generate a calibration curve.

Cell cultures employed in the bioassay can also be provided with the kits according to one embodiment of the invention. In some instances it may be necessary to provide the cells and the remaining components of the kits separately, maintained at different temperatures. In another embodiment, the multi-well plates or test tubes can be “pre-seeded” with the cell culture in a manner known to those skilled in the art, and then shipped for overnight delivery to the user with the other kit components.

Without further description, it is believed that one of ordinary skill in the art can, using the preceding description and the following illustrative examples, make and use the present invention and practice the claimed methods. The following examples are given to illustrate the present invention. It should be understood that the invention is not to be limited to the specific conditions or details described in these examples.

### EXAMPLE 1

### Cell Bank Preparation for Kasumi-1 and M-NFS-60

A vial of Kasumi-1 cell line procured from ATCC is stored in the vapour of liquid nitrogen until revival. The vial is rapidly thawed at 37° C. in a water bath and cells are transferred to 5 mL of RPMI 1640 medium with 20% FBS v/v (complete medium for Kasumi-1 cells). The cells are centrifuged at 25° C., 125 g for 10 minutes. The supernatant is discarded; and the cells are seeded to 5 mL of fresh complete medium in T-25 flasks. The total cell count and % viability is determined by haemocytometer and nucleocounter. Flasks are incubated in 5% v/v CO₂ in air at 37° C., 85% humidity until a high-density culture is obtained. Token, master and working stocks are prepared using 95% of above mentioned complete medium for Kasumi-1 cells and 5% cell culture grade DMSO as a cryoprotectant. Working stock vials are used for all further experimentation and bioassay purpose.

Similarly a vial of M-NFS-60 cell line procured from ATCC is stored in the vapour of liquid nitrogen until revival. The vial is rapidly thawed at 37° C. in a water bath; and the cells are transferred to 5 mL of RPMI 1640 with 10% FBS v/v supplemented with M-CSF 62 ng/mL (complete medium for M-NFS-60 cells). The cells are centrifuged at 25° C., 125 g for 10 minutes. The supernatant is discarded and the cells are seeded to 5 mL of fresh complete medium in T-25 flasks. The total cell count and % viability is determined by haemocytometer and nucleocounter. The flasks are incubated in 5% v/v CO₂ in air at 37° C., and 85% humidity until a high-density culture is obtained. Token, master and working stocks are prepared using 95% of above mentioned complete medium for M-NFS-60 cells and 5% cell culture grade DMSO as a cryoprotectant. Working stock vials are used for all further experimentation and bioassay purpose.

### EXAMPLE 2

### Growth Curves for M-NFS-60 and Kasumi-1

Cryovials of M-NFS-60 and Kasumi-1 cell lines are expanded and sufficient numbers of cells are obtained to start growth curves. Growth curves are set at 1×10⁵ cells/mL for both the cell lines; and a resazurin based redox indicator, Alamar Blue, is used as a growth indicator. RPMI 1640 with 10% FBS and RPMI 1640 with 20% FBS are used as basal media for M-NFS-60 and Kasumi-1, respectively. Both cells are harvested from previous growth flasks by centrifugation at 125 g and 25° C. for 10 minutes in a Sigma cooling centrifuge; and washed thrice with PBS to remove the traces of growth media. The cells are then resuspended in 1 mL of basal media and counted using Chemometec Nucleocounter on 100 μL aliquots. Using these cell suspensions, 20 mL of each cell suspension is prepared at a density of 1×10⁵ cells/mL in respective basal medium.

Alamar Blue is added to the cell suspensions at a concentration of 5% of the final volume. 5 mL of the cell suspension is added to 4 different T-25 flasks for each cell line. For each cell line, rhM-CSF and rhG-CSF are added to 2 of these flasks separately at a final concentration of 62 ng/mL. Both the above cytokines are added at a final concentration of 62 ng/mL to the 3^{rd} flask; and the 4^{th} flask is maintained without any cytokine. All the flasks are incubated in CO₂ incubator at 37° C., 5% CO₂ and 85% RH for next 6 days. During this incubation period, 100 μL samples are withdrawn, in duplicates, from all flasks, and plated in Tarson 96 well microtitre plates at intervals of 0, 24, 48, 72, 96, and 120 hours. Absorbances for these plates are taken using Multiskan EX ELISA reader at 570 nm and 630 nm. Percent reduction of Alamar Blue is calculated for all samples at all intervals (Table 1), as per the calculations given by Alamar Blue product insert from Biosource. The results obtained are analysed by GraphPad software and graphs are plotted in various manner to check the effect of both cytokines on the cell lines in combination and in single use.

TABLE 1
Percentage Reduction of Alamar Blue by M-NFS-60 and Kasumi-1 in Presence
and Absence of M-CSF and G-CSF

| Cytokine | | | | | | | | | | | | |
|---|---|---|---|---|---|---|---|---|---|---|---|---|
| Present in | Incubation Time (hrs.) | | | | | | | | | | | |
| growth | 0 hrs. | | 24 hrs. | | 48 hrs. | | 72 hrs. | | 96 hrs. | | 120 hrs. | |
| medium | M | K | M | K | M | K | M | K | M | K | M | K |
| M-CSF | 1.3 | 4.1 | 30.7 | 12.2 | 49 | 15 | 56.3 | 24.1 | 53.2 | 24.2 | 53.1 | 30.1 |
| (62 ng/mL) | | | | | | | | | | | | |
| G-CSF | 2.1 | 4.6 | 11.5 | 16.5 | 20.3 | 28.3 | 27.7 | 35.3 | 30.3 | 38 | 23.7 | 44.2 |
| (62 ng/mL) | | | | | | | | | | | | |
| M-CSF + | 2.5 | 4.4 | 28.7 | 14.8 | 45.4 | 24.9 | 60.2 | 33.8 | 56.8 | 38.4 | 54.9 | 34.8 |
| G-CSF | | | | | | | | | | | | |
| (62 ng/mL of | | | | | | | | | | | | |
| each) | | | | | | | | | | | | |
| No Cytokine | 3.5 | 4.7 | 19.4 | 9.9 | 14.6 | 26.1 | 18.6 | 24.9 | 15.5 | 24.6 | 24.1 | 46 |

M: - M-NFS-60
K: - Kasumi-1

Analysis of Table 1 gives following observations:

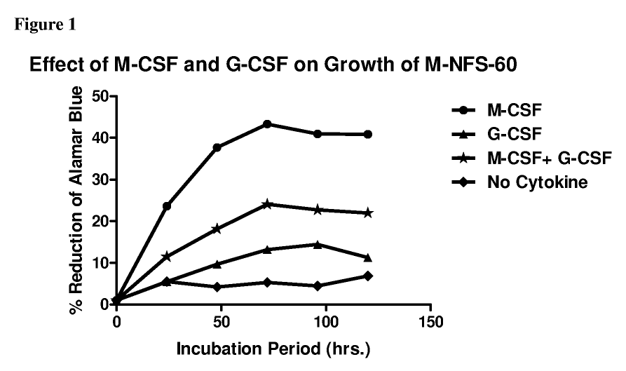

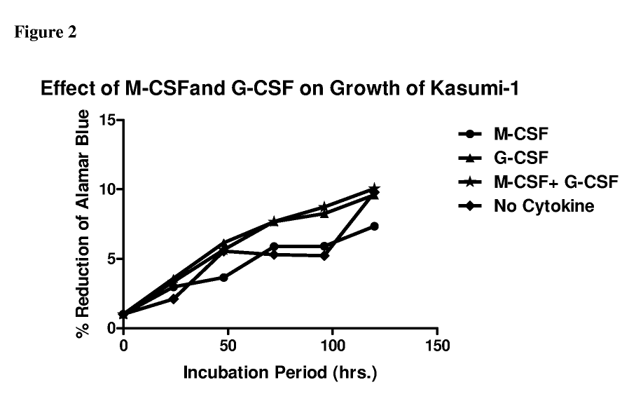

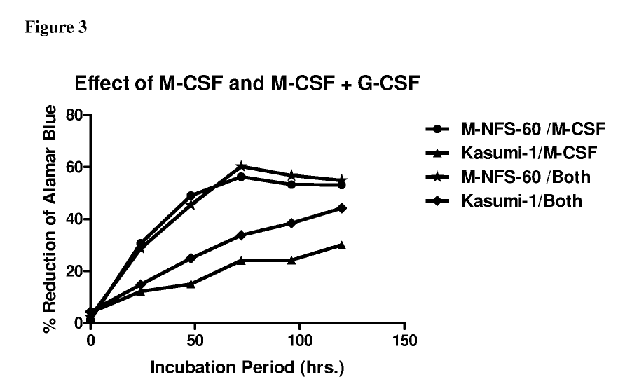

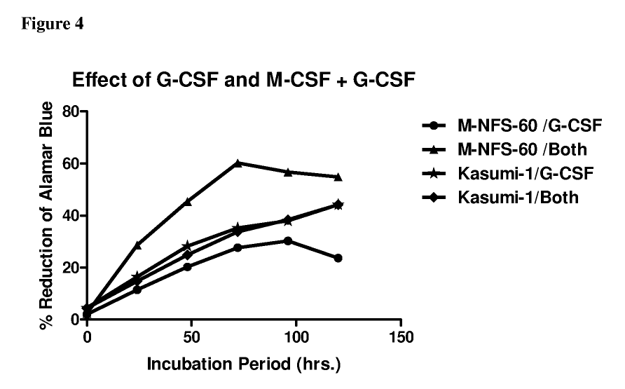

1. Percentage reduction of Alamar Blue by M-NFS-60 cell line in absence of either of cytokine is negligible. M-NFS-60 shows maximum growth in presence of M-CSF and very low growth in presence of G-CSF but absence of M-CSF. Thus the cell line compulsorily requires M-CSF (minimum concentration 62 ng/mL) for maintenance as well as growth even in presence of G-CSF. (FIG. 1)
2. No increase in the Alamar Blue reduction is observed in presence of both cytokines as compared to M-CSF only, which means that response of M-NFS-60 to G-CSF is very weak. (FIG. 3)
3. Considerable reduction of Alamar Blue is observed for Kasumi-1 cell line even in absence of either of the cytokine, which means that none of the cytokine is required for regular maintenance and growth of the cell line. (FIG. 2)
4. Considerable increase in the Alamar Blue reduction is observed when M-CSF is present in the growth medium of M-NFS-60 cells as compared to G-CSF only or no cytokine, which means that response of M-NFS-60 to M-CSF is very strong.
5. Considerable increase in the Alamar Blue reduction is observed when G-CSF is present in the growth medium of Kasumi-1 cells as compared to M-CSF only or no cytokine, which means that response of Kasumi-1 to G-CSF, is very strong. (FIG. 2)
6. No increase in the Alamar Blue reduction is observed in presence of both cytokines as compared to G-CSF only which means that response of Kasumi-1 to M-CSF is very weak. (FIG. 4).

From the above observations, it is clear that M-NFS-60 cell line is strongly responsive to M-CSF and very weakly responsive to G-CSF, whereas Kasumi-1 cell line is strongly responsive to G-CSF and not at all responsive to M-CSF.

### EXAMPLE 3

### Standardisation of Cell Density for G-CSF Bioassay Using Kasumi-1

The following experiment is conducted to determine the optimal cell density of the culture for use with the bioassay.

A stock solution of rhG-CSF is prepared in preincubated sterile Elix water (Elix water is water purified by reverse osmosis and electro deionisation module to obtain deionised water with reduced levels of organic and mineral contaminants) at the concentration of 3 μg/mL. Serial 10 fold dilutions are performed from this stock in sterile Elix water covering a range from 3×10⁻² to 3×10⁶ pg/mL. Kasumi-1 cells are harvested from a culture flask by centrifugation at 25° C., 125 g for 10 minutes. The supernatant is discarded; and the cell pellet is resuspended in 1 mL of preincubated complete medium. The cell density of this suspension is determined by haemocytometer and three cell suspensions are prepared in preincubated complete medium to obtain three different final cell densities of 1×10⁵ cells/mL, 5×10⁵ cells/mL and 1×10⁶ cells/mL. The cell densities of these suspensions are determined by nucleocounter to assure that the cell densities obtained is as desired or adjusted accordingly. 100 μL each of above cell suspensions is plated in 96 well microtitre plate. 50 μL of the each G-CSF dilution is then added to these wells in duplicate. Negative control is maintained for each cell density by adding only media without G-CSF to two wells. The plate is incubated in a CO₂ incubator at 37° C., 5% CO₂ and 85% RH for 72 hrs. At the end of the incubation period, the assay is initiated by adding 15 μL of MTT (Cell Proliferation Kit ATCC Cat. No. 30-1010K) to all the wells; and the plate is again incubated in the CO₂ incubator for 3-4 hrs. At the end of incubation, the purple coloured precipitate of MTT is dissolved by adding 100 μL of detergent (Cell Proliferation Kit ATCC Cat. No. 30-1010K) to all the wells. The plate is incubated at RT in the dark overnight for colour development. Next day absorbance of the plate is taken using Multiskan EX ELISA reader (Labsystems) at 570 nm. The results obtained are analysed by GraphPad Prism software (Version 5.00).

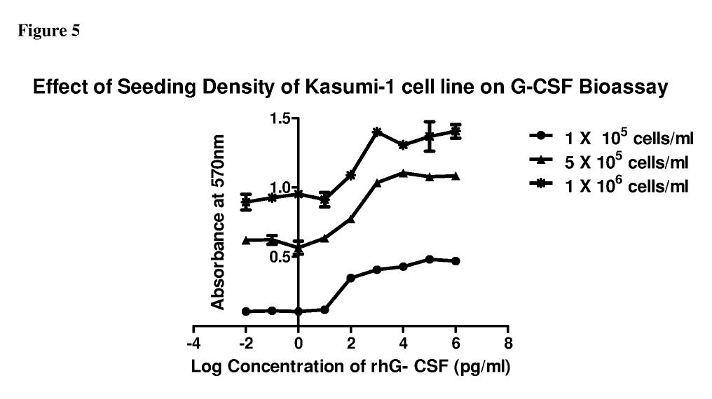

From the results and graphs of above analysis (FIG. 5), it is observed that a cell density of 5×10⁵ cells/mL gives the best results. Readings of 1×10⁵ cells/mL are below and 1×10⁶ cells/mL are above the standard range of the MTT assay kit. Hence, a cell density of 5×10⁵ cells/mL is maintained as the desired cell density for all further experiments.

### EXAMPLE 4

### Standardization of Diluent for G-CSF Bioassay Using Kasumi-1

The diluent used for the dilution of given protein sample can affect the results of bioassay to great extent. Along with proteins, when diluent is added to culture wells, the diluent also dilutes the nutrients of the growth medium and can affect the growth of the indicator cell line. The growth rate and sensitivity of the cell line to various diluents may also be important parameters to consider when selecting a diluent. Hence, the following experiment is conducted to determine the optimal diluent for the G-CSF bioassay.

A stock solution of rhG-CSF is prepared at the concentration of 3 μg/mL in preincubated sterile complete medium and sterile Elix water. Serial 10 fold dilutions are performed from this stock in sterile complete medium and Elix water, respectively, covering a range from 3×10⁻² to 3×10⁶ pg/mL. Kasumi-1 cells are harvested from the culture flask by centrifugation at 25° C., 125 g for 10 minutes. The supernatant is discarded; and the cell pellet is resuspended in 1 mL of preincubated complete medium. The cell density of this suspension is determined by haemocytometer; and a cell suspension is prepared in preincubated complete medium at a density of 5×10⁵ cells/mL. Cell density of this suspension is determined by nucleocounter to assure that cell density is as desired or adjusted accordingly. 100 μL each of above cell suspension is plated in 96 well microtitre plate. 50 μL of the each G-CSF dilution prepared in medium and Elix water is added to these wells separately in duplicates. Negative control is maintained in duplicates for each diluent by adding the respective diluent without G-CSF. The plate is incubated in a CO₂ incubator at 37° C., 5% CO₂ and 85% RH for 72 hrs. At the end of the incubation period, the assay is initiated by adding 15 μL of MTT to all the wells; and the plate is again incubated in the CO₂ incubator for 3-4 hrs. At the end of incubation, the purple coloured precipitate of MTT is dissolved by adding 100 μL of detergent to all wells. The plate is incubated at RT in dark overnight for colour development. Next day absorbance of the plate is taken using Multiskan EX ELISA reader (Labsystems) at 570 nm. The results obtained are analysed by GraphPad software.

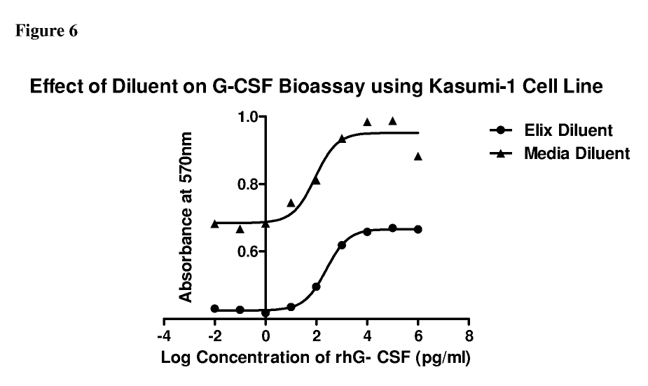

From the results and graphs of above analysis (FIG. 6) it is observed that optical density readings obtained with Elix water dilutions are in the very low range. Elix water, thereby reducing the growth of the Kasumi-1 cells, dilutes the nutrients of the medium. Results of assay with dilutions using complete medium (RPMI-1640) are observed to be in proper range, and hence complete medium (RPMI-1640) is maintained as the diluent for all further experiments.

### EXAMPLE 5

### Standardisation of Incubation Time for G-CSF Bioassay Using Kasumi-1

Incubation time may be a critical parameter in the standardisation of bioassay. As lack of nutrients and increased acidity in media may lead to cell death, ideally an assay should be harvested at an optimal time such that the cells have grown to their maximum limit. The following experiments are performed to determine the optimal incubation time for the G-CSF bioassay.

A stock solution of rhG-CSF is prepared in preincubated sterile complete medium at the concentration of 3 μg/mL. Serial 10 fold dilutions are prepared from this stock in sterile complete medium covering a range from 3×10⁻² to 3×10⁶ pg/mL. Kasumi-1 cells are harvested from the culture flask by centrifugation at 25° C., 125 g for 10 minutes. The supernatant is discarded; and the cell pellet is resuspended in 1 mL of preincubated complete medium. The cell density of this suspension is determined by haemocytometer; and the cell suspension is prepared in preincubated complete medium at a density of 5×10⁵ cells/mL. Cell density of this suspension is determined by nucleocounter to assure that cell density is as desired or adjusted accordingly. 100 μL each of above cell suspension is plated in three separate 96 well microtitre plates. 50 μL of each G-CSF dilution prepared in complete medium is then added to all the plates in duplicate. A negative control is included in each plate, and contains only cells and medium with no G-CSF. All three plates are incubated in a CO₂ incubator at 37° C., 5% CO₂ and 85% RH. A plate is removed at the following time intervals: 24 hrs, 48 hrs, and 72 hrs; and the assay is initiated by adding 15 μL of MTT to all the wells. The plate is again incubated in the CO₂ incubator for 3-4 hrs. At the end of incubation, the purple coloured precipitant of MTT is dissolved by adding 100 μL of detergent to all the wells. The plate is incubated at RT in the dark overnight for colour development. Next day absorbance of the plate is taken using Multiskan EX ELISA reader (Labsystems) at 570 nm. The results obtained are analysed by GraphPad software.

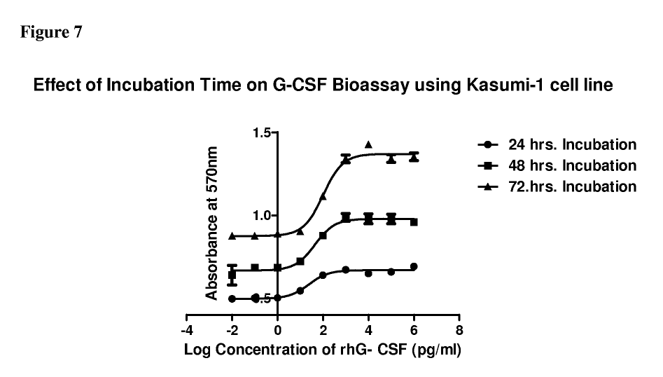

From the results and graphs of above analysis (FIG. 7), it is observed that 24 hrs. incubation is not enough for optimum cell growth as the media is not completely utilised while 72 hrs. incubation results in excessive growth as well as longer duration for determining the assay results. 48 hrs. incubation gives best results within the optimal range. Considering the above results, 48 hrs. incubation is maintained for all further experiments.

### EXAMPLE 6

### Standardisation of Volume of Sample Dilution for G-CSF Bioassay Using Kasumi-1

The volume of the sample dilution being added to each well of microtiter well plate can also be an important parameter because it dilutes the cell concentration in each well. Excess sample volume may reduce the cell density to a large extent leading to inappropriate cell growth rate which in turn may adversely affect the assay result. Thus, the following experiment determines the optimal sample volume to be added to the wells in presence of cells during the G-CSF bioassay.

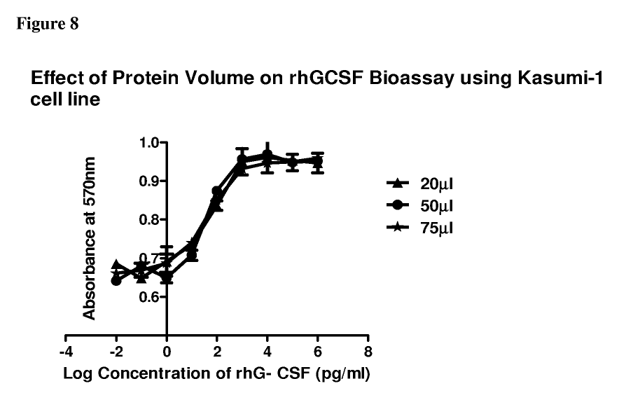

Stock solutions of rhG-CSF are prepared in preincubated sterile complete medium RPMI 1640 with 20% FBS at three different concentrations of 6 μg/mL, 3 μg/mL and 2.33 μg/mL. Serial 10 fold dilutions of above three stocks are performed in sterile complete medium covering a range from 6×10⁻² to 6×10⁶ pg/mL, from 3×10⁻² to 3×10⁶ pg/mL, and from 2.33×10⁻² to 2.33×10⁶ pg/mL respectively. Kasumi-1 cells are harvested from the culture flask by centrifugation at 25° C., 125 g for 10 minutes. The supernatant is discarded; and the cell pellet is resuspended in 1 mL of preincubated complete medium. The cell density of this suspension is determined by haemocytometer; and the cell suspension is prepared in preincubated complete medium at a density of 5×10⁵ cells/mL. The cell density of this suspension is determined by nucleocounter to assure that cell density is as desired. 100 μL of each of the above cell suspensions is plated in a 96-well microtitre plate. 20 μL, 50 μL and 75 μL of above dilutions are then added for 6 μg/mL, 3 μg/mL, and 2.33 μg/mL stock solutions respectively so that actual highest concentration that is obtained in the well is 1 μg/mL. Negative control is included for each sample volume where only medium (of respective volume) is used without G-CSF. The plate is incubated in a CO₂ incubator at 37° C., 5% CO₂, and 85% RH for 48 hrs. At the end of incubation period, the assay is initiated by adding 15 μL of MTT to all the wells, and incubating the plate in the CO₂ incubator for 3-4 hrs. At the end of incubation, the purple coloured precipitate of MTT is dissolved by adding 100 μL of detergent to all wells. The plate is incubated at RT in the dark overnight for colour development. Next day absorbance of the plate is taken using Multiskan EX ELISA reader (Labsystems) at 570 nm. The results obtained are analysed by GraphPad software. From the results and graphs of above analysis (FIG. 8), it is observed that protein volume of 50 μL gives best results hence it is maintained for all further experiments

Table 2 summarizes the results obtained in Examples 1 to Example 6. Thus, in an embodiment of the present invention, the G-CSF bioassay using Kasumi-1 cell line may be optimized using the conditions of Table 2.

TABLE 2
Effect of Various Experimental Parameters on G-CSF Bioassay of Kasumi-1 cell line

| Parameters | | | |
|---|---|---|---|
| Studied | Conditions | Results Obtained | Comments and Conclusion |
| Seeding | 1 × 10⁵ | Readings of 1 × 10⁵ cells/mL | Best results are obtained with |
| Cell | 5 × 10⁵ | and 1 × 10⁶ cells/mL | 5 × 10⁵ cells/mL compared to |
| Density | 1 × 10⁶ | are below and | 1 × 10⁵ cells/mL and 1 × 10⁶ cells/mL |
| (cells/mL) | | above the standard working | |
| | | range of MTT assay kit | |
| Diluent | Elix Water | OD readings with Elix | Elix water dilutes the medium |
| | Complete | dilutions are very low in | components. It hampers the cell |
| | Medium | the range. | growth and hence readings |
| | | | obtained are too low. Complete |
| | | | medium should be used as the |
| | | | diluent. |
| Incubation | 24 hrs. | Cell growth for 24 hrs.is | 24 hrs. incubation is not enough |
| Time | 48 hrs. | not optimum and excessive | to get the maximum cell growth |
| | 72 hrs. | cell growth for 72 hrs. | and hence complete response to |
| | | incubation time. 48 hrs | G-CSF, whereas cell death |
| | | gives optimum growth and | starts in 72 hrs. Hence 48 hrs. is |
| | | best results. | the optimum incubation period |
| Volume of | 20 μL | Best results are obtained | Cell suspension in each well |
| Sample | 50 μL | with 50 μL sample volume. | remains concentrated for 20 μL |
| | 75 μL | | and gets diluted excessively by |
| | | | 75 μL protein dilution. 50 μL of |
| | | | protein dilution gives best |
| | | | results and is the optimum |
| | | | volume. |

### EXAMPLE 7

### Validation of rhG-CSF Bioassay for Kasumi-1 Cell Line and Comparison of rhG-CSF Bioassay Using Kasumi-1 and M-NFS-60 Cell Line

From all the above experiments and permutation combinations of various parameters mentioned above, desired conditions of each parameter are used for further experimental purpose (Table 2). The bioassay of rhG-CSF is performed using those conditions for Kasumi-1 cell line. Any assay format is suitable for performing rhG-CSF bioassay and can be validated as long as the assay selected is relevant, precise and robust. (Mire-Sluis, “Progress in the Use of Biological Assays during the Development of Biotechnology Products,” Pharmaceutical Research, Vol. 18, No. 9, September 2001 pp 1239-1246.)

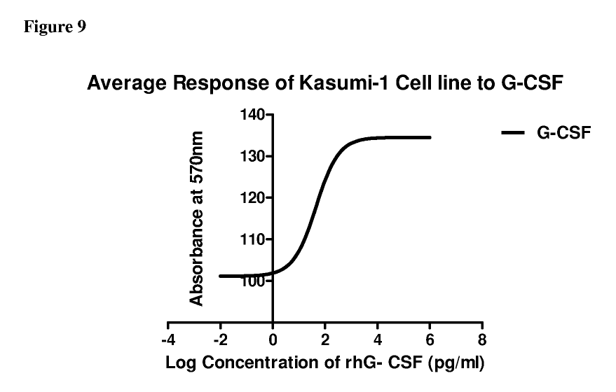

For the validation purpose, the assay is repeated multiple times under identical sets of conditions for above standardised parameters, and the data generated is analysed by GraphPad software for determination of potency in terms of ED₅₀ and specific activity. Also, the absorbance readings are expressed as percentages of negative control i.e. cells without G-CSF and these percentages of multiple replicates of each assay point are averaged for each experiment to get the average response of Kasumi-1 cell line to G-CSF. Coefficient of regression R² is also determined from the average data (Table 3), which always gave a very high value of 0.9 and more. Such high value of R² indicates a very good direct proportion between responses of cells to G-CSF concentration. ANOVA (Analysis of Variance) was also carried out on the data of individual experiments as well as on the average data, which gave a value much more than 0.05 every time. This confirms that the results of G-CSF bioassay performed multiple times in replicates using Kasumi-1 cell line are comparable. (FIG. 9).

TABLE 3
Consistency Determination of G-CSF Bioassay
using Kasumi-1 Cell Line

Potency

| | Stated | Found | |
|---|---|---|---|
| Sample | (IU/mg) | (IU/mg) | R² |
| 1***** | ≧1.0 × 10⁷ | 2.2 × 10⁷ | 0.9948 |
| 2***** | ≧1.0 × 10⁷ | 2.2 × 10⁷ | 0.9666 |
| 3***** | ≧1.0 × 10⁷ | 1.6 × 10⁷ | 0.9882 |

*Number of replicates of sample

To check the specificity of Kasumi-1 cell line towards G-CSF in presence of other cytokines such as M-CSF, a bioassay of G-CSF is performed in presence of M-CSF spiked together.

Stock solution of rhG-CSF and M-CSF are prepared in preincubated sterile complete media at the concentration of 3 μg/ml. Serial 10 fold dilutions are performed from this stock in sterile complete media covering a range of 3×10⁻² to 3×10⁶ pg/ml. Kasumi-1 cells are harvested from the culture flask by centrifugation at 25° C., 125 g for 10 minutes. The supernatant is discarded and the cell pellet is resuspended in 1 ml of preincubated complete media. The cell density of this suspension is determined by haemocytometer and three different cell suspensions are prepared in preincubated complete media without any cytokine (Suspension A), with 100 pg/ml of G-CSF (Suspension B) and with 100 pg/ml of M-CSF (Suspension C), each at a density of 5×10⁵ cells/ml. Cell density of these suspensions is determined by Nucleocounter to assure that cell density adjusted is as desired. 100 μl of cell suspension A is plated in two separate 96 well microtitre plates. Cell suspension B containing G-CSF is plated in one of these plates and cell suspension C containing M-CSF is plated in another plate. 50 μl of the each G-CSF dilution prepared in media is then added to the plate containing cell suspension A and suspension C containing M-CSF at 100 pg/ml concentration. Similarly, 50 μl of the each M-CSF dilution prepared in media is then added to the plate containing cell suspension A and suspension B containing G-CSF at 100 pg/ml concentration. Each dilution is added in duplicates in both the plates. Negative controls are included in each plate containing only cells and media without any cytokine and containing fixed concentration of respective spiked cytokine. Both the plates are incubated in CO₂ incubator at 37° C., 5% CO₂, 85% RH for 48 hrs. At the end of 48 hrs. incubation the plates are harvested by adding 15 μl of MTT to all the wells and the plates are again incubated in CO₂ incubator for 3-4 hrs. At the end of incubation, purple coloured precipitate of MTT is dissolved by adding 100 μl of detergent to all the wells. The plates are incubated at RT in dark for overnight for colour development. Next day absorbance of the plates is taken using Multiskan EX ELISA reader (Labsystems) at 570 nm. Results obtained are analysed by GraphPad software.

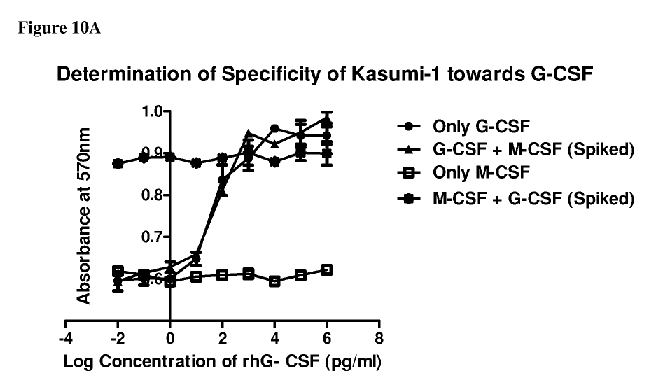

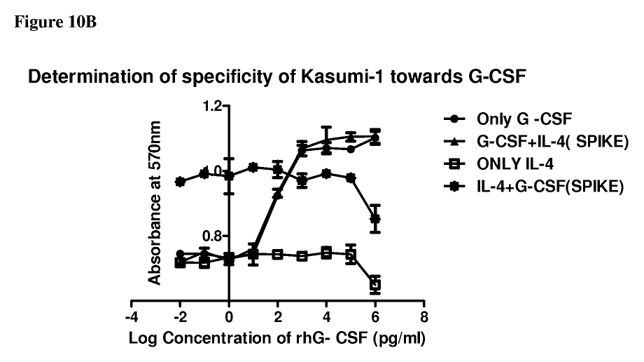

From the results and graphs of the above analysis (FIG. 10A), it is clear that there is no difference in the response of Kasumi-1 cells to G-CSF only (10⁻² to 10⁶ pg/ml) and G-CSF (10⁻² to 10⁶ pg/ml) spiked with M-CSF (10² pg/ml). Results obtained for G-CSF bioassay in both the above conditions are comparable and the ED50 values are in the similar range. But no dose dependent response of Kasumi-1 cells is observed with M-CSF only (10⁻² to 10⁶ pg/ml) and M-CSF (10⁻² to 10⁶ pg/ml) spiked with G-CSF (10² pg/ml). This confirms that Kasumi-1 cells do not respond to M-CSF in a dose dependent manner. Also, the response of cells is much higher, i.e. more cell growth is obtained, when G-CSF is present in the media (spiked at 10² pg/ml) as compared to only media without any cytokine and media containing M-CSF only (Table 4A).

TABLE 4A
Effect of Spiking of M-CSF and G-CSF on Bioassay of Kasumi-1

| | | Results | | | |
|---|---|---|---|---|---|
| | | | Specific | | |
| Experimental | | | Activity | | |
| Condition | Description | Comments | (IU/mg) | R² | Conclusions |
| A | G-CSF (10⁻² | Dose | 2.02 × 10⁷ | 0.9735 | Kasumi-1 responds |
| | to 10⁶ pg/ml) | dependent | | | to G-CSF in dose |
| | | increase in | | | dependent manner |
| | | total cell count | | | |
| | | of Kasumi-1 | | | |
| B | M-CSF | Dose | 1.44 × 10⁷ | 0.9804 | Kasumi-1 responds |
| | spiked | dependent | | | to G-CSF in dose |
| | (10² pg/ml) + | increase in | | | dependent manner |
| | G-CSF | total cell count | | | and shows no |
| | (10⁻² to | of Kasumi-1. | | | increase in readings |
| | 10⁶ pg/ml) | Results similar | | | due to presence of |
| | | to condition A | | | M-CSF. |
| C | M-CSF | No dose | — | — | Kasumi-1 does not |
| | (10⁻² to | dependent | | | respond to M-CSF in |
| | 10⁶ pg/ml) | increase in | | | dose dependent |
| | | total cell count | | | manner. |
| | | of Kasumi-1 | | | |
| D | G-CSF | No dose | — | — | Kasumi-1 does not |
| | spiked | dependent | | | respond to M-CSF in |
| | (10² pg/ml) + | increase in | | | dose dependent |
| | M-CSF | total cell count | | | manner and shows |
| | (10⁻² to | of Kasumi-1. | | | increase in readings |
| | 10⁶ pg/ml) | Absorbance | | | due to spiked G-CSF |
| | | readings much | | | |
| | | higher than | | | |
| | | condition C, E | | | |
| | | and G | | | |
| E | Only M- | Readings | — | — | Kasumi-1 does not |
| | CSF | comparable to | | | show any increase in |
| | (10² pg/ml) | condition C | | | readings due to M- |
| | | and G | | | CSF |
| F | Only G- | Readings | — | — | Kasumi-1 shows |
| | CSF | comparable to | | | increase in readings |
| | (10² pg/ml) | condition D | | | due to G-CSF |
| G | No cytokine | Readings | — | — | Shows normal |
| | (negative | comparable to | | | growth of Kasumi-1 |
| | control) | condition C | | | cells under |
| | | and E | | | experimental |
| | | | | | conditions in |
| | | | | | absence of any test |
| | | | | | reagents. |

These results confirm that Kasumi-1 cell line specifically responds to G-CSF, even in presence of M-CSF, and that M-CSF has no effect on proliferation of Kasumi-1 cells.

To check the specificity of cell line in presence of IL-4, same experiment as mentioned above is repeated using IL-4 instead of M-CSF. All other conditions of experiment are maintained identical as mentioned in the above experiment. The results obtained are analysed by GraphPad software.

From the results and graphs of above analysis (FIGS. 10A and 10B), it is clear that there is no difference in the response of Kasumi-1 cells to G-CSF only (10⁻² to 10⁶ pg/ml) and G-CSF (10⁻² to 10⁶ pg/ml) spiked with IL-4 (10² pg/ml). Results obtained for G-CSF bioassay in above conditions are comparable and the ED₅₀ values are in the similar range. But no dose dependent response of Kasumi-1 cells is observed with IL-4 only and IL-4 (10⁻² to 10⁶ pg/ml) spiked with G-CSF (10² pg/ml). This confirms that Kasumi-1 cells do not respond to IL-4 in dose dependent manner. Also, response of cells is much higher, i.e. more cell growth is obtained when G-CSF is present in the media (spiked at 10² pg/ml), when compared to only media without any cytokine and media containing IL-4 only (Table 4B).

TABLE 4B
Effect of Spiking of IL-4 and G-CSF on Bioassay of Kasumi-1

| | | Results | | | |
|---|---|---|---|---|---|
| | | | Specific | | |
| Experimental | | | Activity | | |
| Condition | Description | Comments | (IU/mg) | R² | Conclusions |
| A | G-CSF (10⁻² | Dose | 1.11 = 10⁷ | 0.9791 | Kasumi-1 responds |
| | to 10⁶ pg/ml) | dependent | | | to G-CSF in dose |
| | | increase in | | | dependent manner |
| | | total cell count | | | |
| | | of Kasumi-1 | | | |
| B | IL-4 spiked | Dose | 1.13 × 10⁷ | 0.9854 | Kasumi-1 responds |
| | (10² pg/ml) + | dependent | | | to G-CSF in dose |
| | G-CSF | increase in | | | dependent manner |
| | (10⁻² to | total cell count | | | and shows no |
| | 10⁶ pg/ml) | of Kasumi-1. | | | increase in readings |
| | | Results similar | | | due to presence of |
| | | to condition A | | | IL-4. |
| C | IL-4 (10⁻² to | No dose | — | — | Kasumi-1 does not |
| | 10⁶ pg/ml) | dependent | | | respond to IL-4 in |
| | | increase in | | | dose dependent |
| | | total cell count | | | manner. |
| | | of Kasumi-1 | | | |
| D | G-CSF | No dose | — | — | Kasumi-1 does not |
| | spiked | dependent | | | respond to IL-4 in |
| | (10² pg/ml) + | increase in | | | dose dependent |
| | IL-4 (10⁻² | total cell count | | | manner and shows |
| | to 10⁶ pg/ml) | of Kasumi-1. | | | higher readings due |
| | | Absorbance | | | to spiked G-CSF |
| | | readings much | | | |
| | | higher than | | | |
| | | condition C, E | | | |
| | | and G | | | |
| E | Only IL-4 | Readings | — | — | Kasumi-1 does not |
| | (10² pg/ml) | comparable to | | | show any increase in |
| | | condition C | | | readings due to IL-4 |
| | | and G | | | |
| F | Only G- | Readings | — | — | Kasumi-1 shows |
| | CSF | comparable to | | | higher readings due |
| | (10² pg/ml) | condition D | | | to G-CSF |
| G | No cytokine | Readings | — | — | Shows normal |
| | (negative | comparable to | | | growth of Kasumi-1 |
| | control) | condition C | | | cells under |
| | | and E | | | experimental |
| | | | | | conditions in |
| | | | | | absence of any test |
| | | | | | reagents. |

These results confirms that Kasumi-1 cell line specifically respond to G-CSF, even in presence of M-CSF and IL-4, and that neither M-CSF nor IL-4 show any effect on Kasumi-1.

To check the robustness of the assay method, effects of various other factors such as position effect in plating, the effect of different sera used to maintain cells and to perform assays, and the effect of different methods of harvesting are also checked.

In vitro assays are prone to position effects that can result in variability in data. To reduce the effects of position within microtitre-plate assays, randomised design of plating is performed. (Mire-Sluis, “Progress in the Use of Biological Assays during the Development of Biotechnology Products,” Pharmaceutical Research, Vol. 18, No. 9, September 2001, pp 1239-1246.)

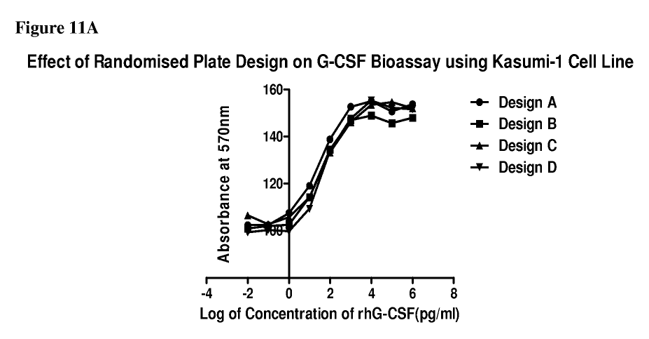

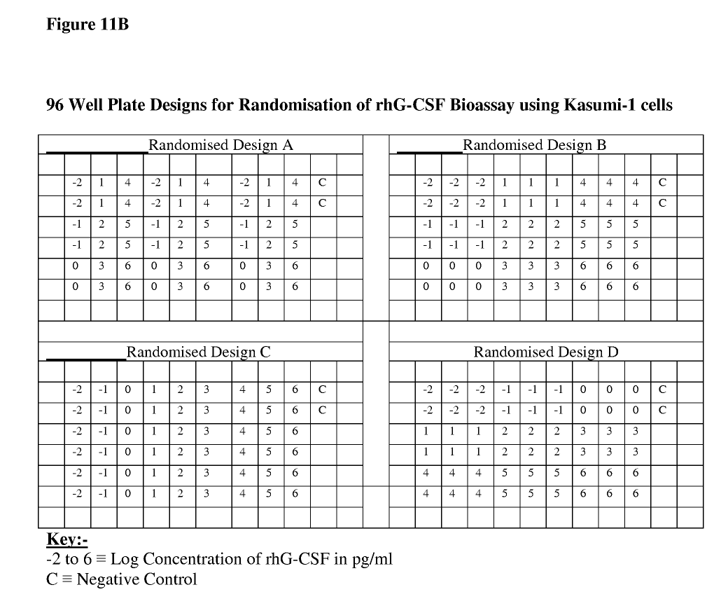

Thus, in order to check the position effect, the assay is repeated using randomised design of plating of rhG-CSF dilutions. Stock solution of rhG-CSF is prepared in preincubated sterile complete media at the concentration of 3 μg/ml. Serial 10 fold dilutions are performed from this stock in sterile complete media covering a range of 3×10⁻² to 3×10⁶ pg/ml. Kasumi-1 cells are harvested from the culture flask by centrifugation at 25° C., 125 g for 10 minutes. The supernatant is discarded and the cell pellet is resuspended in 1 ml of preincubated complete media. The cell density of this suspension is determined by haemocytometer and cell suspension is prepared in preincubated complete media at a density of 5×10⁵ cells/ml. Cell density of this suspension is determined by Nucleocounter to assure that cell density adjusted is as desired. 100 μl of above cell suspension is plated in four separate 96 well microtiter plates. 50 μl of the each G-CSF dilution prepared in media is then added to each plate in three different sets and duplicates of each dilution are added for each set. Four different designs of plating the G-CSF dilutions are used in four different plates (FIG. 11B). Negative control is included in each plate containing only cells and media and no G-CSF. All the plates are incubated in CO₂ incubator at 37° C., 5% CO2, 85% RH. All the plates are removed after 48 hrs. of incubation and harvested by adding 15 μl of MTT to all the wells and the plates are again incubated in CO₂ incubator for 3-4 hrs. At the end of incubation, purple coloured precipitate of MTT is dissolved by adding 100 μl of detergent to all the wells. The plates are incubated at RT in dark for overnight for colour development. Next day absorbance of the plates is taken using Multiskan EX ELISA reader (Labsystems) at 570 nm. Results obtained are analysed by GraphPad software.

The results of the above analysis are graphed in FIG. 11A. Also, the absorbance readings of three different assay designs are expressed as percentages of negative control i.e. cells without G-CSF and average response of cells under particular plate design is determined. The averages of various designs are compared for determination of potency in terms of ED₅₀ and specific activity. Coefficient of regression R² is also determined from the average data (Table 5), which always gave a very high value of 0.9 and more. Such high value of R² indicates a very good direct proportion between responses of cells to G-CSF concentration even under different locations of plating. ANOVA was also carried out on the data of all four randomised designs, which gave a value much more than 0.05 every time. This confirms that the results of G-CSF bioassay performed under various randomised designs of plating do not affect the results. Thus the assay is robust enough to be performed under different plating designs and the results obtained are true response of the cells and are not biased by position of the G-CSF dilutions.

TABLE 5
Effect of Randomised Plate Design on G-CSF Bioassay
using Kasumi-1 Cell Line

Potency

| Randomised | Stated | Found | |
|---|---|---|---|
| Design | (IU/mg) | (IU/mg) | R² |
| A | ≧1.0 × 10⁷ | 3.5 × 10⁷ | 0.9913 |
| B | ≧1.0 × 10⁷ | 3.1 × 10⁷ | 0.9963 |
| C | ≧1.0 × 10⁷ | 1.5 × 10⁷ | 0.9915 |
| D | ≧1.0 × 10⁷ | 1.9 × 10⁷ | 0.9964 |

To check the effect of different sera on the performance of Kasumi-1 cells for G-CSF bioassay, the cells are maintained in two different sera for 2-3 subcultures and the G-CSF bioassay is performed using these cells in complete media containing respective sera.

Two stock solutions of rhG-CSF are prepared in preincubated sterile complete media, one containing Australian origin FBS, JRH-Sigma (Cat. No. 12003) and the other containing European origin FBS, Merck (Cat. No. EU-000-H) at the concentration of 3 μg/ml. Serial 10 fold dilutions are performed from these stocks in sterile complete media containing respective FBS, covering a range of 3×10⁻² to 3×10⁶ pg/ml. Kasumi-1 cells are maintained separately in complete media containing above two different sera for 3 subcultures. These cells are harvested from the culture flasks by centrifugation at 25° C., 125 g for 10 minutes. The supernatant is discarded and the cell pellets are resuspended in 1 ml of respective preincubated complete media. The cell density of both these suspensions are determined by haemocytometer and cell suspensions are prepared in preincubated complete media containing respective sera at a density of 5×10⁵ cells/ml. Cell density of these suspensions are determined by Nucleocounter to assure that cell density adjusted is as desired. 100 μl of above cell suspensions are plated separately in a 96 well microtitre plate. 50 μl of the each G-CSF dilution prepared in respective media is then added to this plate in duplicate. Negative control is included in the plate containing only cells and media with respective sera and no G-CSF. The plate is incubated in CO₂ incubator at 37° C., 5% CO₂, 85% RH for 48 hrs. At the end of incubation the plate is harvested by adding 15 μl of MTT to all the wells and again incubated in CO₂ incubator for 3-4 hrs. At the end of incubation, purple coloured precipitate of MTT is dissolved by adding 100 μl of detergent to all the wells. The plate is incubated at RT in dark for overnight for colour development. Next day absorbance of the plate is taken using Multiskan EX ELISA reader (Labsystems) at 570 nm. Results obtained are analysed by GraphPad software for determination of potency in terms of ED₅₀ and specific activity. Also, averages for duplicates for different G-CSF dilutions are determined and are expressed as percentages of negative control. These percentages are analysed by t-test for determination of p-value by GraphPad software. Coefficient of regression R² is also determined from the average data (Table 6), which always gave a very high value of 0.9 and more. Such high value of R² indicates a very good direct proportion between responses of cells to G-CSF concentration even if the Kasumi-1 cells are maintained in and assayed using sera of different origin and company.

TABLE 6
Effect of Different Sera on G-CSF Bioassay using Kasumi-1
Cell Line

| | |
|---|---|
| Sera used for | Potency |

| Maintenance | Stated | Found | |
|---|---|---|---|
| and Assay | (IU/mg) | (IU/mg) | R² |
| FBS | ≧1.0 × 10⁷ | 1.42 × 10⁷ | 0.9967 |
| Australian | | | |
| (JRH) | | | |
| FBS European | ≧1.0 × 10⁷ | 1.18 × 10⁷ | 0.9737 |
| (Merck) | | | |

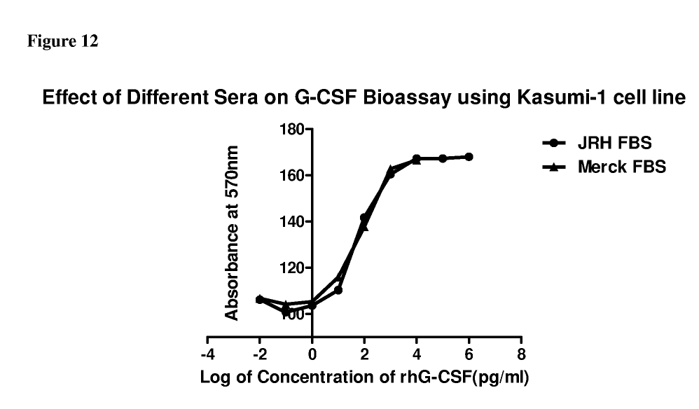

Results and graphs of the above analysis (FIG. 12) show that different sera do not exert any effect on the performance of Kasumi-1 cells for G-CSF bioassay. The cells maintain their morphology and characteristics even when maintained in sera of different origin such as Australian and European and also maintain their responsiveness to G-CSF to same extent as shown by ED₅₀ values (Table 6). Thus, the cells and the assay design is robust enough to be performed using different sera if necessary and the results are still unaffected assuring that the assay can be used in long run experiments where maintenance of cell line in same serum can be difficult to achieve.

To check the effect of different harvesting methods on the G-CSF bioassay of Kasumi-1 cells, the assay is harvested using three different growth indicators such MTT, resazurin based indicator Alamar Blue and LDH.

Stock solution of rhG-CSF is prepared in preincubated sterile complete media at the concentration of 3 μg/ml. Serial 10 fold dilutions are performed from this stock in sterile complete media covering a range of 3×10⁻² to 3×10⁶ pg/ml. Kasumi-1 cells are harvested from the culture flask by centrifugation at 25° C., 125 g for 10 minutes. The supernatant is discarded and the cell pellet is resuspended in 1 ml of preincubated complete media. The cell density of this suspension is determined by haemocytometer and cell suspension is prepared in preincubated complete media at a density of 5×10⁵ cells/ml. Cell density of this suspension is determined by Nucleocounter to assure that cell density adjusted is as desired. 100 μl of above cell suspension is plated in three separate 96 well microtitre plates. 50 μl of the each G-CSF dilution prepared in media is then added to each plate in one set containing duplicates of each dilution. Negative control is included in each plate containing only cells and media and no G-CSF. Appropriate media controls, containing only media and indicator, and media without indicator are also included wherever necessary. All the plates are incubated in CO₂ incubator at 37° C., 5% CO₂, 85% RH. All the plates are removed after 48 hrs. One of these plates is harvested by adding 15 μl of MTT to all the wells and the plate is again incubated in CO₂ incubator for 3-4 hrs. At the end of incubation, purple coloured precipitate of MTT is dissolved by adding 100 μl of detergent to all the wells. The plate is then incubated at RT in dark for overnight for colour development. Next day absorbance of the plate is taken using Multiskan EX ELISA reader (Labsystems) at 570 nm. The second plate is harvested by adding 15 μl of Alamar Blue to all the wells and the plate is again incubated in CO₂ incubator for 3-4 hrs. The absorbance of the plate is then taken using Multiskan EX ELISA reader (Labsystems) at 570 nm and 630 nm. % Reduction of alamar blue is calculated as per the calculations given by AlamarBlue product insert from Biosource. The third plate is harvested by LDH assay using LDH assay kit (Sigma, Cat No. TOX-7) as per the product manual. Averages of the duplicates are calculated for the results obtained by above three harvesting methods and are expressed as percentage of negative control of respective plate. These results obtained are analysed by GraphPad software for determination of potency in terms of ED₅₀ and specific activity. ANOVA is also carried out on the data of all three harvesting methods, which gave a value much more than 0.05. This indicates that there is no difference in the final results of bioassay due to different methods of harvesting. Coefficient of regression R² is also determined from the average data (Table 7), which always gave a very high value of 0.9 and more. Such high value of R² indicates a very good direct proportion between responses of cells to G-CSF concentration even under different methods of harvesting. These results confirms that there is a true response of Kasumi-1 cells to G-CSF in a dose dependent manner which can be elucidated by any of the harvesting methods whether it is mitochondrial reduction based (MTT), metabolic reduction based (Alamar Blue) or cell membrane reduction based (LDH).

TABLE 7
Effect of Different Harvesting Methods on G-CSF Bioassay
using Kasumi-1 Cell Line

Potency

| Harvesting | Stated | Found | |
|---|---|---|---|
| Method | (IU/mg) | (IU/mg) | R² |
| MTT | ≧1.0 × 10⁷ | 3.6 × 10⁷ | 0.9536 |
| Alamar Blue | ≧1.0 × 10⁷ | 3.1 × 10⁷ | 0.9818 |
| LDH | ≧1.0 × 10⁷ | 2.6 × 10⁷ | 0.9918 |

To compare the efficiency of Kasumi-1 and M-NFS-60 cell line for bioassay of G-CSF, the G-CSF bioassay is performed under standardised conditions for both the cell lines.

Stock solution of rhG-CSF is prepared in preincubated sterile complete media and st. Elix at the concentration of 3 μg/ml each. Serial 10 fold dilutions are performed from these stocks in sterile complete media and sterile Elix respectively covering a range of 3×10⁻² to 3×10⁶ pg/ml. Kasumi-1 cells are harvested from the culture flask by centrifugation at 25° C., 125 g for 10 minutes. The supernatant is discarded and the cell pellet is resuspended in 1 ml of preincubated complete media. The cell density of this suspension is determined by haemocytometer and cell suspension is prepared in preincubated complete media at a density of 5×10⁵ cells/ml. Similarly M-NFS-60 cells are harvested from the culture flask by centrifugation at 25° C., 125 g for 10 minutes. The supernatant is discarded and the cell pellet is resuspended in 1 ml of preincubated complete media. The cell density of this suspension is determined by haemocytometer and cell suspension is prepared in preincubated complete media at a density of 2×10⁵ cells/ml. Cell density of both these suspensions is determined by Nucleocounter to assure that cell density adjusted is as desired. 100 μl of each of the above cell suspension is plated in a 96 well microtitre plate separately. 50 μl of the each G-CSF dilution prepared in media is then added to Kasumi-1 cells in duplicates while 50 μl of the each G-CSF dilution prepared in Elix is added to M-NFS-60 cells again in duplicates. Negative control is included for each cell line containing only cells and respective diluent and no G-CSF. The plate is incubated in CO₂ incubator at 37° C., 5% CO2, 85% RH for 48 hrs. At the end of incubation, purple coloured precipitate of MTT is dissolved by adding 100 μl of detergent to all the wells. The plate is incubated at RT in dark for overnight for colour development. Next day absorbance of the plate is taken using Multiskan EX ELISA reader (Labsystems) at 570 nm. Results obtained are analysed by GraphPad software for determination of potency in terms of ED₅₀ and specific activity. Also, averages for duplicates for different G-CSF dilutions are determined and these are analysed by t-test for determination of p-value by GraphPad software.

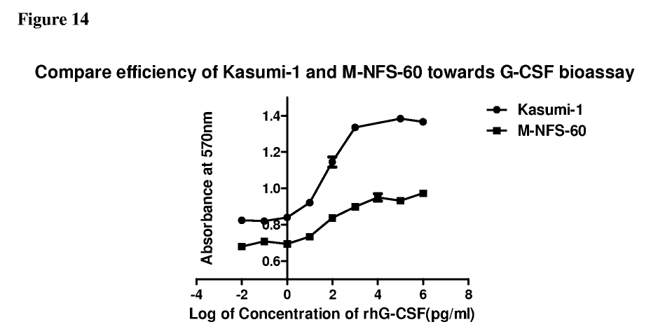

Results and graphs of above analysis (FIG. 14), show that response of Kasumi-1 cells to G-CSF is much better than that of M-NFS-60 cells in terms of ED₅₀ (and hence specific activity) and coefficient of regression R² (Table 8). The increase in absorbance and hence in the cell count shown by Kasumi-1 cells for unit increase in G-CSF concentration is much more than the increase in absorbance and hence in the cell count shown by M-NFS-60 cells for the same increase in G-CSF concentration. Also, the linearity shown by Kasumi-1 cells (R²=0.9954) is much more than the linearity shown by M-NFS-60 cells (R²=0.9679). Thus Kasumi-1 cells gives a more reliable estimate of G-CSF potency in terms of ED₅₀ and specific activity than M-NFS-60 cell line, and should be used as an indicator cell line for potency estimation of G-CSF.

TABLE 8
Compare response of Kasumi-1 and M-NFS-60 cell line for
bioassay of G-CSF,

Potency

| | Stated | Found | |
|---|---|---|---|
| Cell Line | (IU/mg) | (IU/mg) | R² |
| Kasumi-1 | ≧1.0 × 10⁷ | 1.52 × 10⁷ | 0.9954 |
| M-NFS-60 | ≧1.0 × 107 | 1.2 × 10⁷ | 0.9679 |

### EXAMPLE 8

### Application of Validated G-CSF Bioassay Using Kasumi-1 Cell Line

The G-CSF bioassay validated as mentioned in Example 7 is then used for potency estimation of G-CSF samples during the purification of G-CSF and thus used to monitor the efficiency of purification.

rhG-CSF expressed as a recombinant protein in bacterial system is harvested by cell centrifugation. The cells are then lysed by sonication and the protein accumulated in the form of inclusion bodies is separated by centrifugation. The inclusion bodies are solubalised further and refolded. The refolded protein thus obtained is filtered to remove the bacterial cell debris. The filtrate obtained is then concentrated and processed for purification of G-CSF. The concentrated protein is first purified by ion exchange chromatography and then dialysed to remove salts of buffer and other impurities if any. The dialysed sample is further purified by size exclusion chromatography and dialysed again. The dialysed sample thus obtained is then formulated to obtain a stable protein preparation. During this entire process of purification, it is possible that the protein gets denatured or may remain structurally intact but in a biologically inactive form due to presence of various chemicals in the buffers, other reagents of the process or various environmental conditions, such as pH, temperature, etc. Hence, it is necessary to check the activity of the protein sample after every purification step, to assure that purification is progressing correctly without permanent denaturing of the protein.

To check the efficiency of the purification, G-CSF samples are collected at various stages of purification and assayed using bioassay of Kasumi-1 cell line as follows. Protein concentration of various samples is determined by UV absorbance assay and actual G-CSF concentration in each sample is determined by considering the molar extinction coefficient of G-CSF. Stock solutions of various G-CSF samples are prepared in preincubated sterile complete media each at the concentration of 3 μg/ml. Serial 10 fold dilutions are performed from these stock solutions in sterile complete media covering a range of 3×10⁻² to 3×10⁶ pg/ml for each sample. Kasumi-1 cells are harvested from the culture flask by centrifugation at 25° C., 125 g for 10 minutes. The supernatant is discarded and the cell pellet is resuspended in 1 ml of preincubated complete media. The cell density of this suspension is determined by haemocytometer and cell suspension is prepared in preincubated complete media at a density of 5×10⁵ cells/ml. Cell density of this suspension is determined by Nucleocounter to assure that cell density adjusted is as desired. 100 μl of above cell suspension is plated in three separate 96 well microtitre plates. 50 μl of the each G-CSF dilution prepared in media is then added to the plate in duplicates for every sample. Negative control is maintained containing only cells and media and no G-CSF. Similarly to check the effect of buffer of chromatographic process, vehicle control is also included containing cells in media and buffer, wherever necessary. The plates are incubated in CO₂ incubator at 37° C., 5% CO₂, 85% RH. All the plates are removed after 48 hrs and harvested by adding 15 μl of MTT to all the wells and again incubated in CO₂ incubator for 3-4 hrs. At the end of incubation, purple coloured precipitate of MTT is dissolved by adding 100 μl of detergent to all the wells. The plates are then incubated at RT in dark for overnight for colour development. Next day absorbance of the plate is taken using Multiskan EX ELISA reader (Labsystems) at 570 nm. Averages of the duplicates are calculated for the results obtained for various samples. These results are analysed by GraphPad software for determination of potency of each sample in terms of ED₅₀ and specific activity.

Potencies of the various samples are compared to determine the efficiency of the purification process. Coefficient of regression R² is also determined from the average data (Table 10), which always gave a very high value of 0.9 and more. Such high value of R² and almost constant values of potencies of various samples indicates a very good direct proportion between responses of cells to G-CSF concentration and thus ensures that the biological activity of various G-CSF samples from different purification process is unaffected during the entire process of purification.

TABLE 10
Bioassay of Samples from Different Stages of Purification of
G-CSF by using Kasumi-1 Cell Line for Efficiency Check of
Purification Process

| | | Sample | | |
|---|---|---|---|---|
| | Intermediate | Concentration | | |
| Sr. | Stages of | by UV | Potency | |
| No. | Purification | (μg/ml) | (IU/mg) | R² |
| A | After | 1166 | 0.34 × 10⁷ | 0.9968 |
| | Concentration | | | |
| B | After IEX, before | 1031 | 1.8 × 10⁷ | 0.9932 |
| | dialysis | | | |
| C | After IEX, after | 994 | 1.8 × 10⁷ | 0.9852 |
| | dialysis | | | |
| D | After SEC, before | 614 | 1.8 × 10⁷ | 0.9923 |
| | dialysis | | | |
| E | After SEC, after | 528 | 1.7 × 10⁷ | 0.9902 |
| | dialysis | | | |
| F | After Formulation | 331 | 1.8 × 10⁷ | 0.9850 |

These results confirm that various processes of purification are not adversely affecting the biological activity of G-CSF and thus purification of the sample is progressing appropriately.

Although certain presently preferred embodiments of the invention have been specifically described herein, it will be apparent to those skilled in the art, to which the invention pertains that variations and modifications of the various embodiments shown and described herein may be made without departing from the spirit and scope of the invention. Accordingly, it is intended that the invention be limited only to the extent required by the appended claims and the applicable rules of law.

What is claimed is:

1. A method for determining the activity or potency of G-CSF in a sample comprising the steps of
A. contacting the sample with Kasumi-1 cells (ATCC No. CRL 2724);
B. determining the growth of the Kasumi-1 cells; and
C. correlating the growth of the Kasumi-1 cells to an activity of G-CSF.

2. The method of claim 1, wherein step C comprises comparing the growth of the Kasumi-1 cells to a standard curve.

3. The method of claim 1, wherein the Kasumi-1 cells in step A has a density of 1×05 to 1×10⁶ cells/ml.

4. The method of claim 1, wherein the Kasumi-1 cells in step A has a density of 5×10⁵ cells/ml.

5. The method of claim 1, wherein the Kasumi-1 cells are grown in RPMI-1640 medium with 20% FBS.

6. The method of claim 1, wherein step A comprises incubating the sample with the Kasumi-1 cells.

7. The method of claim 6, wherein the incubation takes place at about 37° C. and about 85% relative humidity (RH), with 5% CO₂, for about 24-72 hours.

8. The method of claim 1, wherein the sample is diluted using Elix water or preincubated sterile complete RPMI 1640 media with 20% FBS.

9. The method of claim 1, wherein the sample is diluted using preincubated sterile complete RPMI-1640 media with 20% FBS.

10. The method of claim 1, wherein the sample has a volume of about 20-75 μL.

11. The method of claim 1, wherein the sample has a volume of about 50 μL.

12. The method of claim 1, wherein the Kasumi-1 cells are diluted using preincubated sterile complete RPMI 1640 medium with 20% FBS.

13. The method of claim 1, wherein step B comprises the steps of
i. adding 3-(4,5-dimethylthiazol-2-yl)-2,5-diphenyltetrazolium bromide (MTT) to the mixture of step A;
ii. incubating the mixture of step i for about 3-4 hours;
vi. adding a detergent after the conclusion of step ii;
vii. incubating the mixture of step iii; and
viii. measuring the absorbance of the mixture of step iv at a wavelength of about 500-600 nm.

14. The method of claim 13, wherein the step iv lasts about 16-18 hours.

15. The method of claim 13, wherein step ii occurs at about 37° C., about 5% CO₂, and about 85% relative humidity (RH).

16. The method of claim 13, wherein the wavelength used in step v is 570 nm.

17. The method of claim 1, wherein step C comprises comparing the growth of step B with a standard dose-response curve.

18. The method of claim 1, wherein the sample is diluted before contacting with Kasumi-1 cells.

19. The method of claim 20, wherein the sample is diluted with preincubated sterile complete RPMI 1640 medium with 20% FBS.

20. A kit for carrying out bioassay of G-CSF comprising, in packaged combination,
(a) a first container containing a culture of Kasumi-1 cells;
(b) a second container containing a known amount of G-CSF; and
(c) a third container containing a dilution medium.
